(19) United States
(12) Patent Application Publication
(10) Pub. No.: US 2020/0029928 A1
(43) Pub. Date: Jan. 30, 2020

(54) SYSTEMS AND METHODS FOR IMPROVED MOTION CORRECTION

(71) Applicant: General Electric Company, Schenectady, NY (US)

(72) Inventor: Scott David Wollenweber, Waukesha, WI (US)

(21) Appl. No.: 16/046,649

(22) Filed: Jul. 26, 2018

(51) Int. Cl. A61B 6/00; A61B 6/03; A61B 5/055; A61B 5/00; G06T 11/00

(52) U.S. Cl. CPC A61B 6/5264; A61B 6/032; A61B 5/055; G06T 7/20; A61B 5/7207; G06T 11/003; A61B 6/481; A61B 6/037

(57) ABSTRACT

A method is provided that includes acquiring, with a non-emission imaging acquisition unit, non-emission modality imaging information of an object. The method also includes acquiring, with an emission imaging acquisition unit, emission modality imaging information of the object. Also, the method includes selecting at least one portion of the emission modality imaging information for motion assessment and/or correction based on the non-emission modality imaging information and a clinical task. Further, the method includes performing motion correction on the emission modality imaging information based on the selected at least one portion of the data to provide motion-aware emission modality imaging information, and reconstructing an image using the motion-aware emission modality imaging information.

### BACKGROUND OF THE INVENTION

The subject matter disclosed herein relates generally to imaging systems, and more particularly to methods and systems for selecting portions of information for motion assessment and/or correction.

During operation of medical imaging systems, such as PET imaging systems and/or multi-modality imaging systems (e.g., a PET/Computed Tomography (CT) imaging system, a PET/Magnetic Resonance (MR) imaging system), the image quality may be affected by motion of the object being imaged (e.g., a patient). In particular, motion of the imaged object may create image artifacts during image acquisition, which degrades the image quality. For example, diagnostic confidence may be reduced by the degradation of localization and/or quantification of a tracer-avid feature in an imaging volume caused by movement of the feature. Respiratory motion is an example of a common source of involuntary motion encountered in medical imaging systems.

### BRIEF DESCRIPTION OF THE INVENTION

In an embodiment, a method includes acquiring, with a non-emission imaging acquisition unit, non-emission modality imaging information of an object. The method also includes acquiring, with an emission imaging acquisition unit, emission modality imaging information of the object. Also, the method includes selecting at least one portion of the emission modality imaging information for at least one of motion assessment or motion correction based on the non-emission modality imaging information and a clinical task. Further, the method includes performing at least one of motion assessment or motion correction on the emission modality imaging information based on the selected at least one portion to provide motion-aware (e.g., motion corrected) emission modality imaging information, and reconstructing an image using the motion corrected emission modality imaging information.

In an embodiment, an emission imaging system is provided that includes an emission acquisition unit and at least one processing unit. The emission acquisition unit includes a detector configured to detect emissions from with an object to be imaged. The at least one processing unit is operably coupled to the detector and to the display unit, and is configured to acquire, via a non-emission imaging acquisition unit, non-emission modality imaging information of the object; acquire, with the emission imaging acquisition unit, emission modality imaging information of the object; select at least one portion of the emission modality imaging information for motion correction based on the non-emission modality imaging information and a clinical task; perform at least one of motion assessment or motion correction on the emission modality imaging information based on the selected at least one portion to provide motion corrected emission modality imaging information; and reconstruct an image using the motion corrected emission modality imaging information.

In an embodiment, a tangible and non-transitory computer readable medium is provided that includes one or more computer software modules configured to acquire, via a non-emission imaging acquisition unit, non-emission modality imaging information of the object; acquire, with an emission imaging acquisition unit, emission modality imaging information of the object; select at least one portion of the emission modality imaging information for at least one of motion assessment or motion correction based on the non-emission modality imaging information and a clinical task; perform at least one of motion assessment or motion correction on the emission modality imaging information based on the selected at least one portion to provide motion corrected emission modality imaging information; and reconstruct an image using the motion corrected emission modality imaging information.

### BRIEF DESCRIPTION OF THE DRAWINGS

FIG. 1 is a flowchart of a method for imaging in accordance with various embodiments.

FIG. 2 provides a block diagram of an imaging system in accordance with various embodiments.

FIG. 3 provides a block diagram of a PET imaging system in accordance with various embodiments.

### DETAILED DESCRIPTION OF THE INVENTION

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. For example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated, such as by stating “only a single” element or step. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

“Systems,” “units,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally, or alternatively, one or more of these devices may be hard-wired with logic circuits to perform these operations.

Various embodiments reduce computational requirements, reduce noise, increase image quality, and/or improve the usefulness of motion correction. For example, various embodiments provide for the identification of locations within an imaged volume for which motion assessment and/or correction will be particularly beneficial, and customize or tailor a motion correction for the particular anatomy of a patient and/or for a particular clinical task by using motion correction based upon data from portions of the imaged volume that are of particular clinical interest. Various embodiments provide for pre- or post-scan data localization to enable motion analysis to be more specific to the clinical purpose (or clinical task) of the scan. Various embodiments provide for the tailoring of motion detection, assessment, and/or correction based on features or organs identified within a scanning volume and an associated clinical task (e.g., diagnostic purpose of scan).

Various embodiments provide for improved addressing of motion in emission scanning, for example by selectively using data for and performing a motion correction (and/or motion assessment). A technical benefit of at least one embodiment includes reduction in noise and/or improvement in image quality by eliminating or reducing unnecessary or undesirable data utilized for a motion correction (and/or motion assessment).

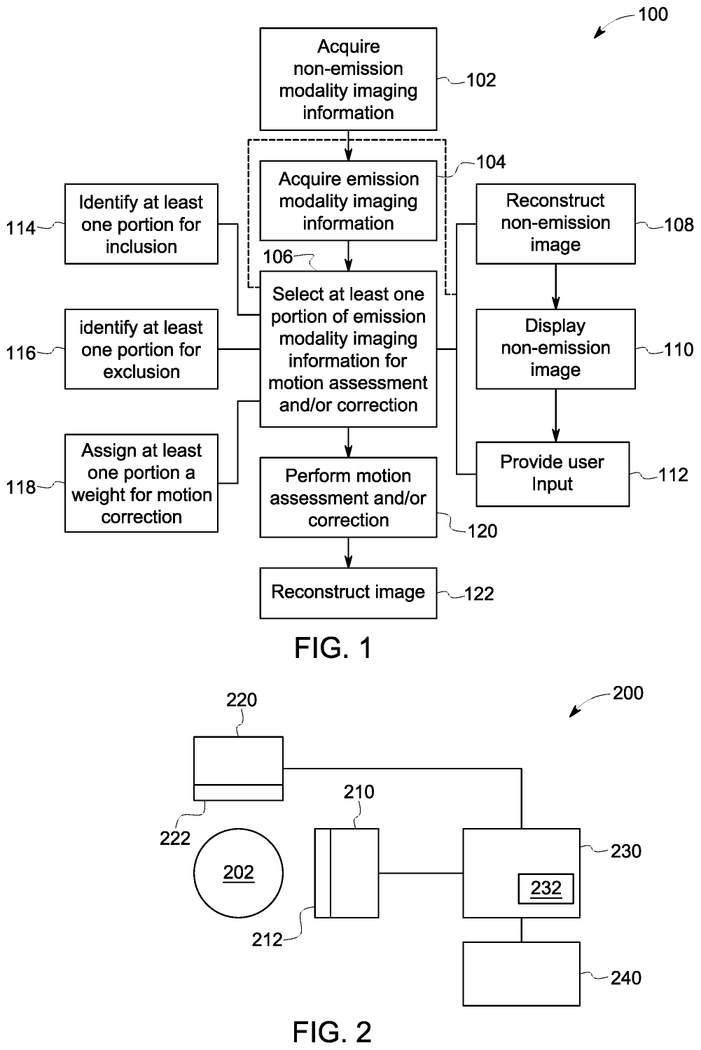

FIG. 1 provides a flowchart of a method 100 for imaging an object, in accordance with various embodiments. The method 100, for example, may employ or be performed by structures or aspects of various embodiments (e.g., systems and/or methods and/or process flows) discussed herein. In various embodiments, certain steps may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 100 may be able to be used as one or more algorithms to direct hardware (e.g., one or more processing units such as processing unit 230, and/or one or more processing units including one or more aspects of the motion correction module 494) to perform one or more operations described herein.

At 102, non-emission modality imaging information is acquired of an object (e.g., human patient or portion thereof). Generally, the non-emission modality imaging information is acquired using an imaging technique that does not utilize radiative emissions that originate from within the object. The non-emission modality imaging information may, for example, include computed tomography (CT) imaging information acquired using a CT acquisition unit or imaging system that includes an X-ray source and detector that rotates about the object while acquiring the CT imaging information. Alternatively or additionally, the non-emission modality imaging information may include, for example, magnetic resonance (MR) imaging data acquired with an MR acquisition unit or imaging system. Generally, in various embodiments, a sufficient amount of the non-emission modality imaging information is acquired to provide sufficient structural detail so that particular structures of interest (e.g., particular organs and/or features such as tumors or lesions) may be identified.

At 104, emission modality imaging information of the object is acquired with an emission imaging acquisition unit or imaging system. Generally, the emission modality imaging information is acquired using an imaging technique that utilizes radiative emissions that originate from within the object. For example, a patient may be administered with a radiopharmaceutical, with the uptake of the radiopharmaceutical in different portions of the patient resulting in different levels or amounts of emissions from the different portions, with the levels or amounts of emissions from the different portions detected and used to reconstruct an image corresponding to the uptake of the radiopharmaceutical and resulting emissions. It may be noted that the volume imaged for the emission modality imaging information overlaps partially or entirely with the volume imaged for the non-emission modality imaging information, so that locations within the emission modality imaging information corresponding to features or structures (e.g., organs or tumors) identified in the non-emission imaging modality imaging information may be identified.

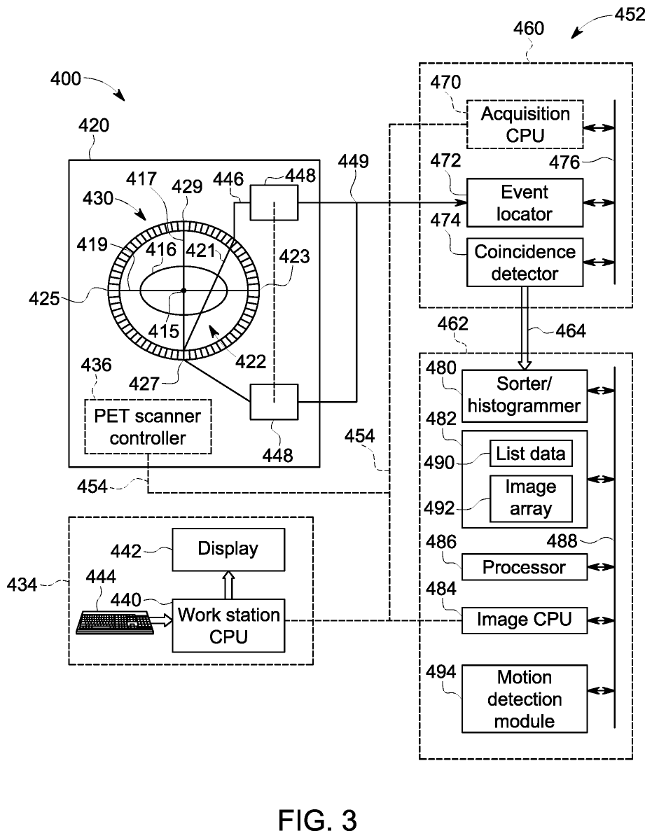

In some embodiments, positron emission tomography (PET) imaging data (e.g., coincidence data) is acquired with a PET acquisition unit or imaging system. The imaging data may be acquired, for example, using a PET detector (see, e.g., FIG. 3), and may be stored in a list of events ('list-mode'). In some embodiments, list-mode data may be stored for each coincident event (e.g., each event corresponding to detection by opposed portions of a PET detector, or each paired event), with a position (e.g., x and y), a time, and an energy level stored in list-mode for each coincident event. The detector used to acquire the PET imaging data defines a field of view (FOV) for the acquired imaging volume. For example, the FOV may be made up of positional FOV's from a plurality of bed positions. The detector may acquire information at a first bed position over a first FOV, at a second bed position over a second FOV, and so on. The FOV defined by the detector for the entire scan or acquisition may be determined by combining the positional FOV's from each bed position (accounting for any overlap between positional FOV's as appropriate). In some embodiments, the method 100 may be performed for each bed or detector position separately, with the FOV defined as the positional FOV for the given bed or detector position.

In various embodiments, the PET imaging data may be acquired using a cylindrical detector including rings of detector elements, with the detector advanced relative to a bed or table (e.g., the detector may be advanced and the bed or table fixed, or the bed or table may be advanced and the detector fixed) along an axial length of an object (e.g., human patient) to be imaged. The detector may be advanced in a step-and-shoot manner, in which the detector is advanced to a given detector or bed position along the axial length of an object, the detector is then stopped, information is acquired while the detector is stopped, and, following a desired amount of time for information acquisition, the detector is then advanced to one or more subsequent detector positions differently located along the axial length of the object to be imaged. Alternatively, the detector may be advanced continuously along the length of the object in some embodiments. This is functionally equivalent to continually advancing the object to be imaged relative to the detector. It may be noted that, while certain embodiments discussed herein are discussed in connection with PET, that other types or modalities of imaging data may be acquired or utilized additionally or alternatively in various embodiments. For example, in some embodiments, single photon emission computed tomography (SPECT) imaging data may be acquired. PET imaging data may be understood as one example of emission modality imaging information, emission imaging data, or emission data. It may also be noted that the emission modality imaging information and non-emission modality imaging information may be acquired with a multi-modality system including both emission and non-emission acquisition modalities, or may be acquired by separate systems (e.g., a patient may be imaged separately using one more emission modality systems and one or more non-emission modality systems located in the same building, department, or facility, for example).

It may further be noted that the list-mode data discussed above may be down-sampled (sampled more coarsely in space or time) or otherwise used to generate sinogram information. A sinogram in PET is a 2D data representation using line-of-response distance from axis and angle as the two coordinates. A set of sinograms is typically generated as a sorted histogram of events in (r, theta, z) format and is most commonly used as a precursor of image reconstruction. A set of sinograms over time may also be formed. As used herein, “sinogram” is a general term implying counts of activity along Lines Of Response (LORs) of a detector. In general, the format of the segment data in various embodiments may be in one or more of a multitude of formats, including but not limited to sinograms, projection view data, Singles histograms, or coincidence event histograms, and is not necessarily restricted to a specific dimensional data set.

At 106, at least one portion of the emission modality imaging information is selected for motion assessment and/or correction based on the non-emission modality imaging information and a clinical task. Generally, in various embodiments, the non-emission imaging information is used to identify locations in the shared imaging volume having features of particular interest (e.g., organs and/or tumors) for motion correction. Features that are particularly susceptible to motion and/or features of relatively higher diagnostic value for a given clinical task may be selected for motion assessment and/or correction, whereas features that have relatively lower diagnostic value may be omitted from motion correction. In various embodiments, particular types of organs or other structures pertinent to a clinical task are identified using the non-emission modality imaging information, and corresponding locations in the emission modality imaging information are selected for motion assessment and/or correction based on the identified organs or other structures. It may further be noted that the at least one portion for motion assessment and/or correction may be selected prospectively (e.g., before acquiring the emission modality imaging information), or retrospectively (e.g., after acquiring the emission modality imaging information).

In some embodiments, at least one portion of the emission modality imaging information may be selected for motion assessment and/or correction based on or using a user input. For example, in the example illustrated in FIG. 1, at 108 a non-emission image (e.g., a CT image) is reconstructed using the non-emission modality imaging information. At 110, the non-emission image is displayed, for example to a practitioner. At 112, a user input is provided (and received by a processing unit) that describes or corresponds to one or more structures (e.g., organs and/or other features such as tumors) and/or locations within the imaging volume. The selections indicated by the user input may then be used to selectively perform motion correction. The user input 112 in various embodiments may be provided, for example, via a touchscreen or other interactive display. Further, in various embodiments, the user may be provided with information regarding the clinical task and/or a listing of structures or features of particular interest to the clinical task to guide the selection to help guide the user input. In alternate embodiments, at least one portion of the emission modality imaging information may be selected for motion assessment and/or correction autonomously or automatically using one or more processing units programmed to perform the selection.

As stated above, in various embodiments, certain steps may be performed in a different order than as shown for the particular illustrative example of FIG. 1. As one example, the series of steps performed at 108-110-112 (or aspects thereof) may be performed between steps 102 and 104. This is represented in FIG. 1 by a dashed line. Similarly, the selection of the at least one portion for motion correction shown in step 106 as occurring after the acquisition of emission modality imaging information in step 104 may be performed in various embodiments before step 104. This is also represented schematically with a dashed line in FIG. 1.

It may be noted that one or more portions of the imaging volume or of the emission modality imaging information may be selected by inclusion and/or exclusion. For example, only selected portions may be assessed for motion and motion corrected, while un-selected portions may not be motion corrected or included in the motion assessment. As another example, the imaging volume in general may be motion corrected except for specific portions identified to not be motion corrected.

In the illustrated embodiment, at 114, at least one portion is identified for inclusion. For instance, for a clinical task that requires or benefits from a detailed inspection of the imaged lungs, the lungs may be identified based on the non-emission modality imaging information, and the corresponding locations within the emission modality imaging information selected for motion correction. Also in the illustrated embodiment, at 116, at least one portion is identified for exclusion. For instance, for a clinical task that requires or benefits from a detailed inspection of the lungs but does not require or generally benefit from analysis of the kidneys, the kidneys may be identified based on the non-emission modality imaging information, and the corresponding locations within the emission modality imaging information omitted from a motion assessment and/or correction.

It may further be noted that the selection of a location or locations within the imaging volume of the emission modality imaging information need not be selected in a strictly 100% included or 100% excluded fashion. For example, in some embodiments, as part of a motion correction, one or more portions of data are assigned weights, with higher weights corresponding to a greater impact on the motion assessment and/or motion correction. In the illustrated embodiment, at 118, at least one portion of the data is assigned at least one weight to be used in a motion assessment and/or correction. For example, an organ or organs of particular interest or diagnostic value for a particular clinical task may be weighted relatively highly, while an organ or organs of less interest or diagnostic value for the particular clinical task may be weighted relatively low (or at zero), so that motion assessment and/or correction is more focused on the organ or organs of particular interest or greater diagnostic value. Similarly, a feature such as a tumor identified using the non-emission modality imaging information may be assigned a further weight for motion assessment and/or correction that may differ from that used for the above-discussed organs. Accordingly, features or structures within an imaging volume may be provided hierarchically-ranked in terms of diagnostic value and/or susceptibility to motion, and a corresponding variety of weights may be assigned for motion assessment and/or correction for the feature or structures based on the diagnostic value and/or susceptibility to motion.

At 120, motion assessment and/or correction is performed on the emission modality imaging information based on the selected at least one portion (e.g., the at least one portion selected at 106) to provide motion-aware emission modality imaging information. Accordingly, by selectively performing motion assessment and/or correction for particular locations of interest within an imaging volume, the motion assessment and/or correction may be performed more efficiently or effectively. As the particular locations are selected using imaging information from the particular patient for which an emission modality image will be reconstructed, the motion assessment and/or correction may be customized or tailored for the particular patient (e.g., based on the location of particular structures of interest within the patient based on the non-emission modality imaging information) and particular clinical task (or diagnostic purpose), providing improved motion assessment and/or correction and overall, improved imaging. For example, for a given clinical task, both the lungs and kidneys may be subject to movement, but the lungs may be of particular interest for diagnostic purposes for the given clinical task, whereas the kidneys may of lesser interest or diagnostic usefulness for the given clinical task. Accordingly, the locations of the imaging volume for the patient corresponding to the lungs may be identified using the non-emission modality imaging information and selected for motion correction, while the location corresponding to the kidneys may not be selected for motion correction.

It may be noted that the motion assessment and/or correction in various embodiments is performed using a variational analysis. For example, principle components analysis (PCA) may be utilized to perform the motion assessment prior to a motion correction. It may be noted that PCA is an example of a multivariate data analysis technique, and that other multivariate data analysis techniques may be employed additionally or alternatively in various embodiments. Examples of multivariate data analysis techniques include PCA, independent component analysis (ICA), and regularized PCA (rPCA).

At 122, an image is reconstructed using the motion corrected emission modality imaging information. The image may be displayed or otherwise provided to a practitioner for performing a diagnosis pursuant to the clinical task.

FIG. 2 depicts an imaging system 200 formed in accordance with various embodiments. The depicted imaging system 200 includes an emission acquisition unit 210, a non-emission acquisition unit 220, a data-processing unit 230, and a display unit 240. Generally, the components (individually and/or collectively) may be utilized to acquire imaging information, perform selective motion processing as discussed herein, and reconstruct an image (e.g., to perform one or more aspects of the method 100).

The depicted emission acquisition unit 210 includes a detector 212 configured to detect emissions (e.g., emissions resulting from an administered radiopharmaceutical) from with an object 202 (e.g., a human patient or portion thereof) to be imaged. The emission acquisition unit 210 is configured to obtain emission modality imaging information of the object 202. The emission acquisition unit 210, for example, may include or be configured as a PET detection unit or a SPECT detection unit. (See also FIG. 3 and related discussion for an example PET detector system.)

The depicted non-emission acquisition unit 220 includes a detector 222 configured to detect or collect non-emission modality imaging information from the object 202. For example, the non-emission acquisition unit 220 may include or be configured as a CT acquisition unit or a MR acquisition unit. It may be noted that the non-emission modality imaging information may be acquired before, after, or concurrently with the emission modality imaging information. In some embodiments, the emission acquisition unit 210 and the non-emission acquisition unit 220 may both be included as part of a multi-modality system. As another example, the non-emission acquisition unit 220 may be separate from the emission acquisition unit 210 but located in same room, facility, or department for convenient imaging of the same object 202 (or portion thereof) as imaged by the emission acquisition unit 210.

The depicted processing unit 230 is operably coupled to the emission acquisition unit 210 (e.g., to the detector 212) and to the non-emission acquisition unit 220 (e.g., to the detector 222). In various embodiments the processing unit 230 includes processing circuitry configured to perform one or more tasks, functions, or steps discussed herein (e.g., in connection with the method 100 or aspects thereof). It may be noted that “processing unit” as used herein is not intended to necessarily be limited to a single processor or computer. For example, the processing unit 230 may include multiple processors, ASIC's and/or computers, which may be integrated in a common housing or unit, or which may distributed among various units or housings. It may be noted that operations performed by the processing unit 230 (e.g., operations corresponding to process flows or methods discussed herein, or aspects thereof) may be sufficiently complex that the operations may not be performed by a human being within a reasonable time period.

In the illustrated embodiment, the processing unit 230 includes a memory 232 that stores a set of instructions to direct the processing unit 230 to perform one or more aspects of the methods, steps, or processes discussed herein. For example, in various embodiments the processing unit 230 is configured to acquire non-emission modality information via the non-emission imaging acquisition unit 220 and to acquire emission modality information with the emission imaging acquisition unit 210. The processing unit 230 may also be configured to select at least one portion of the emission modality imaging information for motion assessment and/or correction based on the non-emission modality imaging information and a clinical task, perform motion correction on the emission modality imaging information based on the selected at least one portion to provide motion corrected emission modality imaging information, and reconstruct an image using the motion corrected emission modality imaging information.

The depicted display unit 240 is coupled to the processing unit 230 and configured to display images and/or information provided from the processing unit 230, and/or to receive a user input to provide information or direction to the processing unit 230. For example, the display unit 240 in various embodiments includes a touchscreen configured for interactive display of a reconstructed image (e.g., an image reconstructed using the non-emission modality imaging information) and receipt of user inputs identifying locations and/or features for motion correction selection. Alternatively or additionally, the display unit 240 may also be utilized to display a motion corrected emission image for diagnostic use.

Various example scenarios, which may employ or utilize one or more aspects of the method 100 and/or the system 200 will now be discussed. In a first example scenario, CT or MM is used to explicitly include or exclude organs of interest during a prospective motion correction. In the first example scenario, a user interface is presented to a technologist prior to emission scanning that allows for explicit inclusion or exclusion of a certain organ (or organs) within a prospective motion correction method (such as prospective data-driven gating (DDG) followed by quiescent-period gating (Q.Static) PET). For example, prior to molecular or emission imaging (e.g., PET or SPECT), a patient is scanned with CT or MM. The CT or MM imaging information is segmented to determine organ localizations. By way of example, segmentation methods may include (but are not necessarily limited to) threshold-based organ segmentation; machine learning (ML) based methods to determine organ localization, and/or allowing a user to apply a weight to the organ, allowing the organ to differentially impact the motion assessment. Next, the location(s) of the affected organ(s) (e.g., within the bed positions of a PET scan) are determined. When scanning a bed position that contains an included or excluded organ, a projection of the organ may be used to explicitly include or exclude that organ projection data as part of the down-sampled sinogram (DSS) information from the emission modality. A PCA analysis for motion assessment using the DSS information will then explicitly include or exclude data related to motion of that particular organ (or organs).

In a second example scenario, CT or MRI is used to explicitly include or exclude one or more features of interest (e.g., tumor, lesion, lung nodule) during a prospective motion assessment and/or correction. For example, prior to molecular or emission imaging (e.g., PET or SPECT), a patient is scanned with CT or MM. A user interface is presented to the technologist (e.g., including an image reconstructed using information from the CT or MM scan) prior to the molecular or emission imaging scan that allows for explicit inclusion or exclusion of one or more features within a prospective motion assessment and/or correction method (such as prospective DDG+Q.Static PET). The user may interactively select an area or areas in the CT or MRI image volume for inclusion (or exclusion) in the motion assessment. In another embodiment, the user may apply weights to features, making the corresponding data more likely (or less likely, depending on the applied weight) to impact a motion assessment and/or correction of the data. Next, the location of the feature(s) may be determined within the bed position of a PET scan. When scanning a bed position that contains a user-defined feature, a projection of the feature may be used to explicitly or with-weighting include that feature's projection data as part of the DSS data. A PCA analysis for motion assessment then explicitly includes or excludes data related to the user-defined feature(s).

In a third example scenario, CT or MRI is used to explicitly include or exclude organs of interest during a retrospective motion assessment and/or correction. In the third example scenario, a user interface is presented as part of a post-processing method to a technologist that allows for explicit inclusion or exclusion of a certain organ (or organs) from a motion assessment and/or correction method (such as DDG+Q, Static PET). A CT or MRI image is then segmented to determine organ localizations. Segmentation methods may include (but are not necessarily limited to) threshold-based organ segmentation or ML based methods to determine organ localization. Optionally, for example, a user may apply a weight to the organ, allowing the organ to differentially impact the motion assessment. Next, the location(s) of the affected organ(s) (e.g., within the bed positions of a PET scan) are determined. When determining the motion for a bed position that contains an included or excluded organ, a projection of the organ may be used to explicitly include, exclude or with-weighting include that organ as part of the DSS information. A PCA analysis for motion assessment using the DSS information will then explicitly include or exclude data related to motion of that particular organ (or organs).

In a fourth example scenario, CT or MRI is used to explicitly include or exclude a feature (or features) of interest during a retrospective motion correction. As a post-processing method, a user inter may be presented to a technologist that allows for explicit inclusion or exclusion of one or more features within a motion correction method (such as DDG+Q. Static PET). For example, a user may interactively select an area or areas in a CT or MRI image volume. Optionally, the user may assign a weight to a feature (or features), making the feature more likely to impact a motion assessment of the data in the FOV or imaging volume. Next it may be determined where the feature(s) are located within the bed positions of a PET scan. A projection of the feature(s) may be used to explicitly include or with-weighting include the feature as part of the DSS data when determining the motion for a bed position. Accordingly, a resulting PCA analysis for motion assessment then explicitly includes or excludes data related to the user-defined features in the fourth example scenario.

It may be noted that the above four scenarios are presented by way of example. Other variations may be utilized in various embodiments. Generally, under any of the scenarios, more information about a clinical indication (e.g., an identification of an existing tumor using CT imaging information) or about an area of most interest in a scan (e.g., an emphasis on lesions in the liver and a disregard or lowered emphasis on kidney motion) may be used to tune or tailor a data-driven motion assessment and subsequent correction method.

Various methods and/or systems (and/or aspects thereof) described herein may be implemented using a medical imaging system. For example, FIG. 3 is a block schematic diagram of an exemplary PET imaging system 400 that may be utilized to implement various embodiments discussed herein. The PET imaging system 400 may be used to acquire PET coincidence event data during a PET scan. The PET imaging system 400 includes a gantry, an operator workstation 434, and a data acquisition subsystem 452. In a PET scan, a patient 416 is initially injected with a radiotracer. The radiotracer comprises bio-chemical molecules that are tagged with a positron emitting radioisotope and can participate in certain physiological processes in the body of the patient 416. When positrons are emitted within the body, the positrons combine with electrons in the neighboring tissues and annihilate, creating annihilation events. The annihilation events usually result in pairs of gamma photons, with 511 keV of energy each, being released in opposite directions. The gamma photons are then detected by a detector ring assembly 430 within the gantry 420 that includes a plurality of detector elements (e.g., 423, 425, 427, 429). The detector elements may include a set of scintillator crystals arranged in a matrix that is disposed in front of a plurality of photosensors such as multiple photo multiplier tubes (PMTs) or other light sensors. When a photon impinges on the scintillator of a detector element, the photon produces a scintillation (e.g., light) in the scintillator. Each scintillator may be coupled to multiple photo multiplier tubes (PMTs) or other light sensors that convert the light produced from the scintillation into an electrical signal. In addition to the scintillator-PMT combination, pixilated solid-state direct conversion detectors (e.g., CZT) may also be used to generate electrical signals from the impact of the photons.

The detector ring assembly 430 includes a central opening 422, in which an object or patient, such as the patient 416 may be positioned, using, for example, a motorized table (not shown). The scanning and/or acquisition operation is controlled from an operator workstation 434 through a PET scanner controller 436. Typical PET scan conditions include data acquisition at several discrete table locations with overlap, referred to as ‘step-and-shoot’ mode. Optionally, during the PET scan, the motorized table may traverse through the central opening 422 while acquiring PET coincidence event data, for example, a continuous table motion (CTM) acquisition. The motorized table during the CTM acquisition may be controlled by the PET scanner controller 436. During the CTM acquisition, the motorized table may move through the central opening 422 at a consistent or stable velocity (e.g., within a predetermine velocity threshold during the PET scan).

A communication link 454 may be hardwired between the PET scanner controller 436 and the workstation 434. Optionally, the communication link 254 may be a wireless communication link that enables information to be transmitted to or from the workstation 434 to the PET scanner controller 436 wirelessly. In at least one embodiment, the workstation 434 controls real-time operation of the PET imaging system 400. The workstation 434 may also be programmed to perform medical image diagnostic acquisition in reconstruction processes described herein.

The operator workstation 434 includes a work station central processing unit (CPU) 440, a display 442 and an input device 444. The CPU 440 connects to a communication link 454 and receives inputs (e.g., user commands) from the input device 444, which may be, for example, a keyboard, a mouse, a voice recognition system, a touch-screen panel, or the like. Through the input device 444 and associated control panel switches, the clinician can control the operation of the PET imaging system 400. Additionally or alternatively, the clinician may control the display 442 of the resulting image (e.g., image-enhancing functions), physiologic information (e.g., the scale of the physiologic waveform), the position of the patient 416, or the like, using programs executed by the CPU 440.

During operation of the PET imaging system, for example, one pair of photons from an annihilation event 415 within the patient 416 may be detected by two detectors 427 and 429. The pair of detectors 427 and 429 constitute a line of response (LOR) 417. Another pair of photons from the region of interest 415 may be detected along a second LOR 419 by detectors 423 and 425. When detected, each of the photons produce numerous scintillations inside its corresponding scintillators for each detector 423, 425, 427, 429, respectively. The scintillations may then be amplified and converted into electrical signals, such as an analog signal, by the corresponding photosensors of each detector 423, 425, 427, 429.

A set of acquisition circuits 448 may be provided within the gantry 420. The acquisition circuits 448 may receive the electronic signals from the photosensors through a communication link 446. The acquisition circuits 448 may include analog-to-digital converters to digitize the analog signals, processing electronics to quantify event signals, and a time measurement unit to determine time of events relative to other events in the system 400. For example, this information indicates when the scintillation event took place and the position of the scintillator crystal that detected the event. The digital signals are transmitted from the acquisition circuits 448 through a communication link 449, for example, a cable, to an event locator circuit 472 in the data acquisition subsystem 452.

The data acquisition subsystem 452 includes a data acquisition controller 460 and an image reconstruction controller 462. The data acquisition controller 460 includes the event locator circuit 472, an acquisition CPU 470 and a coincidence detector 474. The data acquisition controller 460 periodically samples the signals produced by the acquisition circuits 448. The acquisition CPU 470 controls communications on a back-plane bus 476 and on the communication link 454. The event locator circuit 472 processes the information regarding each valid event and provides a set of digital numbers or values indicative of the detected event. For example, this information indicates when the event took place and the position of the scintillator crystal that detected the event. An event data packet is communicated to the coincidence detector 474 through a communication link 476. The coincidence detector 474 receives the event data packets from the event locator circuit 472 and determines if any two of the detected events are in coincidence.

Coincidence may be determined by a number of factors. For example, coincidence may be determined based on the time markers in each event data packet being within a predetermined time period, for example, 12.5 nanoseconds, of each other. Additionally or alternatively, coincidence may be determined based on the LOR (e.g., 417, 419) formed between the detectors (e.g., 423 and 425, 427 and 429). For example, the LOR 417 formed by a straight line joining the two detectors 427 and 429 that detect the PET coincidence event should pass through a field of view in the PET imaging system 400. Events that cannot be paired may be discarded by the coincidence detector 474. PET coincidence event pairs are located and recorded as a PET coincidence event data packet that is communicated through a physical communication link 464 to a sorter/histogrammer circuit 480 in the image reconstruction controller 462.

The image reconstruction controller 462 includes the sorter/histogrammer circuit 480. During operation, the sorter/histogrammer circuit 480 generates a PET list data 490 or a histogram, which may be stored on the memory 482. The term “histogrammer” generally refers to the components of the scanner, e.g., processor and memory, which carry out the function of creating the PET list data 490. The PET list data 490 includes a large number of cells, where each cell includes data associated with the PET coincidence events. The PET coincidence events may be stored in the form of a sinogram based on corresponding LORs within the PET list data 490. For example, if a pair of PET gamma photons are detected by detectors 427 and 429, the LOR 417 may be established as a straight line linking the two detectors 427 and 429. This LOR 417 may be identified as two dimensional (2-D) coordinates (r, θ, Δt), wherein r is the radial distance of the LOR from the center axis of the detector ring assembly 430, θ is the trans-axial angle between the LOR 417 and the X-axis, and At is the change in time of the detection of the photons between the two detectors 427 and 429 of the LOR 417. The detected PET coincidence events may be recorded in the PET list data 490. As the PET scanner 436 continues to acquire PET coincidence events along various LORs (e.g., 417, 419, 421), these events may be binned and accumulated in corresponding cells of the PET list data 490. The result is a 2-D sinogram λ(r, θ, Δt), each of which holds an event count for a specific LOR. In another example, for a three dimensional (3-D) sinogram, an LOR 417, 419 may be defined by four coordinates (r, θ, z, Δt), wherein the third coordinate z is the distance of the LOR from a center detector along a Z-axis.

Additionally, the communication bus 488 is linked to the communication link 454 through the image CPU 484. The image CPU 484 controls communication through the communication bus 488. The array processor 486 is also connected to the communication bus 488. The array processor 486 receives the PET list data 490 as an input and reconstructs images in the form of image arrays 492. Resulting image arrays 492 are then stored in a memory module 482. The images stored in the image array 492 are communicated by the image CPU 484 to the operator workstation 434.

The PET imaging system 400 also includes a motion correction module 494. The depicted motion correction module 494 is configured to perform one or more aspects, steps, operations or processes discussed herein (e.g., in connection with the method discussed in connection with FIG. 1). For example, the motion correction module 494, and/or other aspect(s) of a processing unit, may be configured to identify one or more portions of emission modality imaging information using non-emission modality imaging information and a clinical task, and perform the motion assessment and/or correction.

The depicted motion correction module 494 may include one or more aspects of processing unit 230 in various embodiments, and is an example of a processing unit configured to perform one or more tasks or operations disclosed herein. As discussed herein, a processing unit as used herein may include processing circuitry configured to perform one or more tasks, functions, or steps discussed herein. It may be noted that “processing unit” as used herein is not intended to necessarily be limited to a single processor or computer. For example, a processing unit may include multiple processors and/or computers, which may be integrated in a common housing or unit, or which may distributed among various units or housings.

It should be noted that the particular arrangement of components (e.g., the number, types, placement, or the like) of the illustrated embodiments may be modified in various alternate embodiments. For example, in various embodiments, different numbers of a given module or unit may be employed, a different type or types of a given module or unit may be employed, a number of modules or units (or aspects thereof) may be combined, a given module or unit may be divided into plural modules (or sub-modules) or units (or sub-units), one or more aspects of one or more modules may be shared between modules, a given module or unit may be added, or a given module or unit may be omitted.

As used herein, a structure, limitation, or element that is “configured to” perform a task or operation may be particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a processing unit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optic drive, and the like. The storage device may also be other similar means for loading computer programs or other instructions into the computer or processor.

As used herein, the term “computer,” “controller,” “system,” and “module” may each include any processor-based or microprocessor-based system including systems using microcontrollers, reduced instruction set computers (RISC), application specific integrated circuits (ASICs), logic circuits, GPUs, FPGAs, and any other circuit or processor capable of executing the functions described herein. The above examples are exemplary only, and are thus not intended to limit in any way the definition and/or meaning of the term “module” or “computer.”

The computer, module, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, module, or processor as a processing machine to perform specific operations such as the methods and processes of the various embodiments described and/or illustrated herein. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph unless and until such claim limitations expressly use the phrase “means for” followed by a statement of function void of further structure.

This written description uses examples to disclose the various embodiments, and also to enable a person having ordinary skill in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

The foregoing description of certain embodiments of the present inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general-purpose signal processor, microcontroller, random access memory, hard disk, or the like). Similarly, the programs may be stand alone programs, may be incorporated as subroutines in an operating system, may be functions in an installed software package, or the like. The various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “comprises,” “including,” “includes,” “having,” or “has” an element or a plurality of elements having a particular property may include additional such elements not having that property.

What is claimed is:

1. A method comprising:
acquiring, with a non-emission imaging acquisition unit, non-emission modality imaging information of an object;
acquiring, with an emission imaging acquisition unit, emission modality imaging information of the object;
selecting at least one portion of the emission modality imaging information for at least one of motion assessment or correction based on the non-emission modality imaging information and a clinical task;
performing at least one of motion assessment or correction on the emission modality imaging information based on the selected at least one portion to provide motion-aware emission modality imaging information; and
reconstructing an image using the motion-aware emission modality imaging information.

2. The method of claim 1, wherein selecting the at least one portion comprises identifying portions to be included in the at least one of motion assessment or correction.

3. The method of claim 1, wherein selecting the at least one portion comprises identifying portions to be excluded from the at least one of motion assessment or correction.

4. The method of claim 1, wherein selecting the at least one portion comprises assigning at least one weight to the at least one portion to be used in the at least one of motion assessment or correction.

5. The method of claim 1, wherein the selecting the at least one portion is performed before acquiring the emission modality imaging information.

6. The method of claim 1, wherein the selecting the at least one portion is performed after acquiring the emission modality imaging information.

7. The method of claim 1, wherein the selecting the at least one portion comprises displaying a non-emission image reconstructed using the non-emission imaging information, and receiving a user input corresponding to the displayed non-emission image.

8. An emission imaging system comprising:
an emission acquisition unit comprising a detector configured to detect emissions from with an object to be imaged; and
at least one processing unit operably coupled to the detector and to the display unit and configured to
acquire, via a non-emission imaging acquisition unit, non-emission modality imaging information of the object;
acquire, with the emission imaging acquisition unit, emission modality imaging information of the object;
select at least one portion of the emission modality imaging information for at least one of motion assessment or correction based on the non-emission modality imaging information and a clinical task;
perform at least one of motion assessment or correction on the emission modality imaging information based on the selected at least one portion to provide motion-aware emission modality imaging information; and
reconstruct an image using the motion-aware emission modality imaging information.

9. The emission imaging system of claim 8, wherein the at least one processing unit is configured to select the at least one portion by identifying portions to be included in the at least one of motion assessment or correction.

10. The emission imaging system of claim 8, wherein the at least one processing unit is configured to select the at least one portion by identifying portions to be excluded from the at least one of motion assessment or correction.

11. The emission imaging system of claim 8, wherein the at least one processing unit is configured to assign at least one weight to the at least one portion to be used in the at least one of motion assessment or correction.

12. The emission imaging system of claim 8, wherein the at least one processing unit is configured to select the at least one portion before acquiring the emission modality imaging information.

13. The emission imaging system of claim 8, wherein the at least one processing unit is configured to select the at least one portion after acquiring the emission modality imaging information.

14. The emission imaging system of claim 8, wherein the at least one processing unit is configured to display a non-emission image reconstructed using the non-emission imaging information, receive a user input corresponding to the displayed non-emission image, and use the user input to select the at least one portion of the data.

15. A tangible and non-transitory computer readable medium comprising one or more computer software modules configured to direct one or more processors to:
acquire, via a non-emission imaging acquisition unit, non-emission modality imaging information of the object;
acquire, with an emission imaging acquisition unit, emission modality imaging information of the object;
select at least one portion of the emission modality imaging information for at least one of motion assessment or correction based on the non-emission modality imaging information and a clinical task;
perform at least one of motion assessment or correction on the emission modality imaging information based on the selected at least one portion to provide motion-aware emission modality imaging information; and
reconstruct an image using the motion-aware emission modality imaging information.

16. The tangible and non-transitory computer readable medium of claim 15, wherein the computer readable medium is further configured to direct the one or more processors to select the at least one portion by identifying portions to be included in the at least one of motion assessment or correction.

17. The tangible and non-transitory computer readable medium of claim 15, wherein the computer readable medium is further configured to direct the one or more processors to select the at least one portion by identifying portions to be excluded from the at least one of motion assessment or correction.

18. The tangible and non-transitory computer readable medium of claim 17, wherein the computer readable medium is further configured to direct the one or more processors to assign at least one weight to the at least one portion to be used in the at least one of motion assessment or correction.

19. The tangible and non-transitory computer readable medium of claim 15, wherein the computer readable medium is further configured to direct the one or more processors to select the at least one portion before acquiring the emission modality imaging information.

20. The tangible and non-transitory computer readable medium of claim 15, wherein the computer readable medium is further configured to display a non-emission image reconstructed using the non-emission imaging information, receive a user input corresponding to the displayed non-emission image, and use the user input to select the at least one portion of the data.
